(19) United States
(12) Patent Application Publication
(10) Pub. No.: US 2006/0002911 A1
(43) Pub. Date: Jan. 5, 2006

(54) Association between a ppar ligand and an antioxidant agent and use thereof for treating obesity

(76) Inventors: Louis Casteilla, Escalquens (FR); Luc Penicaud, Toulouse (FR); Catherine Dacouet, Paris (FR); Pierre Renard, Le Chesnay (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/530,771

(22) PCT Filed: Oct. 10, 2003
(86) PCT No.: PCT/FR03/02986

§ 371 Date: Apr. 7, 2005

(30) Foreign Application Priority Data
Oct. 11, 2002 (FR) 02/12646

(51) Int. Cl. A61K 38/43; A61K 31/426

(52) U.S. Cl. 424/94.1; 514/369

(57) ABSTRACT

The invention relates to a combination comprising one or more ligands of peroxisome proliferator activated receptors and an antioxidant agent, and to pharmaceutical compostions comprising the combination.

The present invention relates to the association between one or more selective ligands of peroxisome proliferator activated receptors (PPAR) and an antioxidant agent and to the use thereof in the treatment and/or prevention of obesity and of overweight characterised by a body mass index greater than 25.

Obesity is a major public health problem in all developed countries. It is also increasing steadily in developing countries and is affecting an ever younger population. Obesity is a chronic disorder of energy imbalance characterised by an excess of energy intake in the long term compared with limited energy expenditure, leading to storage of the excess energy in the form of white adipose tissue.

Excess adipose tissue directly contributes to problems of fatigue, shortness of breath, sleep apnoea and osteoarthritis.

Obesity is a well-established risk factor for the development of insulin resistance, of dyslipidaemia and, ultimately, of non-insulin-dependent diabetes. It is a factor contributing to cardiovascular diseases and is associated with a significantly increased risk of cerebro-vascular accidents, vesicular calculi, respiratory dysfunction, osteoarthritis, several forms of cancer and premature death.

A pharmacological strategy for reducing obesity presents two alternatives: either to reduce fat by modifying energy intake and/or by modifying the distribution of nutrients between fat and lean tissues, or to counter or reverse the metabolic consequences of the increase in fat without necessarily having an impact on the degree of obesity in itself.

It has been found that, in obese people, the generation of reactive oxygenated species released by monocytes and leukocytes is greatly increased with respect to non-obese subjects (J. Clin. Endocrinol. Metab., 2001, 86, 355-362). Elevated plasma concentrations of alpha tumour necrosis factor (TNFα) in obese people stimulate inflammatory processes (J. Clin. Endocrinol. Metab., 1998, 83, 2907-2910) and are responsible for the generation of reactive oxygenated species by leukocytes (Oncogene, 1998, 17, 1639-1651).

The pathological state of obesity is also associated with increased oxidation of lipids and proteins, which may be the cause of high plasma levels of 9- and β-hydroxy-octadecadienoic acids (9-HODE and 13-HODE) (Totowa: Humano. Press., 1998, 147-155), key indices of lipid peroxidation (J. Clin. Endocrinol. Metab., 2001, 86, 355-362). In parallel, the “antioxidant” capabilities of the body are reduced.

In obese subjects, it has been shown that excessive food intake causes major lipid and protein damage. Over-consumption of calories by obese people can cause the formation of free radicals and expose them to significant oxidative lesions which help to maintain the state of obesity.

The specific markers of oxidation are significantly reduced by a 48-hour fast or by calorie restriction accompanying weight loss (J. Clin. Endocrinol. Metab., 2001, 86, 355-362).

A strategy aimed at reducing the “oxidative burden” on the body by favouring the lipid and carbohydrate metabolisms should result in an exacerbation of the effects and, as a consequence, in weight loss in obese or overweight subjects.

Among the compounds capable of favouring the lipid and carbohydrate metabolisms, selective ligands of peroxisome proliferator activated receptors or PPARs are especially interesting compounds.

The PPARs are a family of nuclear hormone receptors comprising three distinct sub-types: α, β (also called δ or NUC1) and γ (which has three isoforms: γ₁, γ₂ and γ₃).

They were initially cloned as nuclear receptors mediating the effects of peroxisome proliferators on gene transcription, but it is clear that a very large number of natural compounds such as eicosanoids and fatty acids can also activate PPARs.

Like a certain number of other nuclear hormone receptors, PPAR proteins bind to promoters in the form of heterodimers with the 9-cis-retinoic acid receptor, RXR. The PPAR/RXR heterodimer can be activated by the binding of a ligand specific to one of the two receptors but maximum activation is achieved when two ligands are present.

PPARs are ligand-dependent transcription factors, which means that inititation of transcription of the target genes is strictly dependent on the binding of the ligand.

Certain ligands, such as mono- or poly-unsaturated fatty acids or saturated fatty acids, bind to the three sub-types of receptor. Long-chain polyunsaturated fatty acids, such as linolenic acid, or oxidated or conjugated fatty acids bind to PPARα with a high degree of affinity.

The most important function of PPARs results from their tissue-dependent expression and from their specific target genes which are very often involved exclusively in the transport and metabolism of lipids and carbohydrates.

The PPARα KO mouse develops obesity and hypertriglyceridaemia even if the daily intake of calories is not increased. These effects are largely explained by a reduction in fatty acid uptake by the liver and inhibition of fatty acid oxidation (J. Biol. Chem., 1998, 273, 29577-29585).

The liver is an organ capable of oxidising fatty acids. When hepatic oxidation of fatty acids is optimal, thermogenesis comes into play and converts the available energy into heat, with a reduction in the respiratory quotient and an increase in the basic metabolic rate. These circumstances are highly favourable to the loss of adipose tissue (Med. Hypotheses, 1999, 52(5), 407-416).

A strategy consisting of disinhibiting the enzymatic processes of hepatic oxidation of fatty acids whilst ensuring transcriptional stimulation of genes activated by PPARs and involved in lipid and carbohydrate metabolic processes should result in a reduction in free fatty acids in the plasma and in moderated lipolysis in adipocytes constituting visceral adipose tissue, in the long term bringing about a regression in visceral obesity and, accordingly, a reduction in body weight.

The present invention relates, more specifically, to the association between one or more compounds that are ligands of peroxisome proliferator activated receptors and an antioxidant agent.

This association exhibits pharmacological properties that are entirely remarkable in the area of obesity.

More specifically, the PPAR ligands according to the invention are selective ligands for the α and/or γ receptor sub-types.

Advantageously, the association according to the invention comprises a selective PPARα ligand and a selective PPARγ ligand.

An advantageous embodiment relates to the association according to the invention wherein the PPAR ligand is a mixed ligand of the α and γ receptor sub-types.

PPARα and/or γ ligands according to the invention are advantageously represented by gemfibrozil, WY-14,643, pioglitazone and, even more preferably, by rosiglitazone.

PPAR ligands according to the invention are also represented by the compounds described in the Applications WO 9736579, WO 9731907, WO9728115, WO9638415, WO9727857, WO9725042, WO9701420, WO9640128, WO2000064888 and WO2000064876.

Antioxidant agents according to the invention are represented by various categories of compound:
anti-free radical agents or free-radical trapping agents,
antilipoperoxidant agents,
chelating agents,
agents capable of regenerating endogenous antioxidants such as glutathione, vitamin C or vitamin E.

The antioxidant agent of the association according to the invention is more preferably represented by quinone compounds such as ubiquinone or coenzyme Q₁₀, which acts as a free-radical trapping agent but which is also capable of regenerating vitamin E.

The enantiomers and diastereoisomers and addition salts with a pharmaceutically acceptable acid or base of the PPAR ligand and antioxidant compounds according to the association likewise form an integral part of the invention.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc.

Amongst the pharmaceutically acceptable bases there may be mentioned, without implying any limitation, sodium hydroxide, potassium hydroxide, triethylamine, tert-butylamine, etc.

The association to which preference is given in accordance with the invention is rosiglitazone and coenzyme Q₁₀.

Furthermore, the association according to the invention between one or more compounds favouring the lipid and carbohydrate metabolisms and an antioxidant agent has entirely surprising pharmacological properties: the Applicant has discovered that a synergy exists between those two classes of compound allowing a very significant reduction in body fat to be obtained, making it of use in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin-resistance, dyslipidaemia and the appearance of macrovascular disorders (nephropathy, retinopathy, angiopathy). Further pathologies are the consequence of obesity or overweight: there may be mentioned, in particular, vesicular calculi, respiratory dysfunction, osteoarthritis, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25 and less than 30.

The invention accordingly relates to the use of the association comprising one or more PPAR ligands and an antioxidant agent in obtaining pharmaceutical compositions intended for the treatment and/or prevention of obesity and of overweight characterised by a body mass index greater than 25 and less than 30.

In particular, the association according to the invention is of use in the treatment and/or prevention of obesity and of overweight characterised by a body mass index greater than 25 and less than 30 caused by a therapeutic treatment, such as treatment for type I or II diabetes.

The invention accordingly relates to the use of an association comprising one or more PPAR ligands and an antioxidant agent in obtaining pharmaceutical compositions intended for the treatment and/or prevention of obesity and of overweight characterised by a body mass index greater than 25 and less than 30 caused by a therapeutic treatment, such as treatment for type I or II diabetes.

The invention relates also to pharmaceutical compositions comprising the association between one or more compounds favouring the lipid and carbohydrate metabolisms, more especially one or more PPAR ligands, and an antioxidant agent, as defined hereinbefore, in combination with one or more pharmaceutically acceptable excipients.

Among the pharmaceutical compositions according to the invention, there may be mentioned, more especially, those that are suitable for oral, parenteral or nasal administration, tablets or dragées, sublingual tablets, gelatin capsules, lozenges, suppositories, creams, ointments, dermal gels, etc.

The dosage used varies according to the sex, age and weight of the patient, the administration route, the nature of the therapeutic indication or of any associated treatments and ranges from 0.1 mg to 1 g of each component of the association per 24 hours in one or more administrations.

The Examples that follow illustrate the invention but do not limit it in any way.

### EXAMPLE A

### Chance in Body Weight

Male C57 Black 6 ob/ob mice from 8 to 12 weeks old were used. The mice are diabetic (type II) and suffer from hyperglycaemia, hypertriglyceridaemia and hyperinsulinaemia. After being placed in quarantine for one week, they were weighed and then randomised as a function of their weight and 6 homogeneous groups (starting weights not significantly different) were formed. After being weighed, the mice were treated with rosiglitazone (antidiabetic agent) on its own or in association with coenzyme Q₁₀. The compounds were injected by the intraperitoneal route once a day for 14 days in a solution of DMSO 5%/Solutol 15%/qsp H₂O heated at 65° C. to ensure good dissolution. In addition, the solution was pre-heated before injection. The mice were weighed every day and the weight obtained after 14 days of treatment was recorded.

Treatment with rosiglitazone alone results in an increase in the weight of the mice greater than or equal to 5 grams, corresponding to about 10% more than their initial weight. The association rosiglitazone+coenzyme Q₁₀ allows that weight gain to be reversed by at least 50% and demonstrates the effectiveness of the association in reducing body weight.

### EXAMPLE B

### Pharmaceutical Composition

100 tablets each containing 30 mg of rosiglitazone and 10 mg of coenzyme Q₁₀

| | |
|---|---|
| Rosiglitazone | 3 g |
| Coenzyme Q₁₀ | 1 g |
| Wheat starch | 20 g |
| Maize starch | 20 g |
| Lactose | 30 g |
| Magnesium stearate | 2 g |
| Silica | 1 g |
| Hydroxypropylcellulose | 2 g |

1-25. (canceled)

26. A combination comprising a combination of an antioxidant agent and a mixed PPAR ligand for the α and γ receptor sub-types, or a selective PPAR ligand for the α receptor sub-type and a selective PPAR ligand for the γ receptor sub-type.

27. The composition of claim 26, wherein the PPAR ligand is a mixed ligand for the α and γ receptor sub-types.

28. The composition of claim 26, comprising an antioxidant agent and a selective ligand for the α receptor sub-type and a selective ligand for the γ receptor sub-type.

29. The composition of claim 26, wherein the selective PPAR ligand for the γ receptor sub-type is rosiglitazone or an addition salt thereof with a pharmaceutically acceptable acid or base.

30. The composition of claim 26, wherein the antioxidant agent is ubiquinone or coenzyme Q₁₀.

31. A composition comprising a combination of rosiglitazone and coenzyme Q₁₀.

32. A pharmaceutical composition comprising as active ingredient a composition of claim 26, in combination with one or more pharmaceutically-acceptable excipients.

33. A method for treating and/or preventing a living animal body, including a human, afflicted with obesity comprising the step of administering to the living animal body, including a human, an amount of a composition of claim 26 which is effective for alleviation of obesity.

34. A method for treating a living animal body, including a human, afflicted with obesity caused by a therapeutic treatment comprising the step of administering to the living animal body, including a human, an amount of a composition of claim 26 which is effective for alleviation of said disorder.

35. A method for treating a living animal body, including a human, afflicted with obesity caused by treatment for type I or II diabetes comprising the step of administering to the living animal body, including a human, an amount of a compostion of claim 26 which is effective for alleviation of obesity caused by treatment for type I or II diabetes.

36. A method for treating and/or preventing a living animal body, including a human, afflicted with an overweight condition characterised by a body mass index greater than 25 and less than 30 comprising the step of administering to the living animal body, including a human, an amount of a composition of claim 26 which is effective for alleviation of the condition.

37. A method for treating a living animal body, including a human, afflicted with an overweight condition characterised by a body mass index greater than 25 and less than 30 caused by a therapeutic treatment comprising the step of administering to the living animal body, including a human, an amount of a composition of claim 26 which is effective for alleviation of the condition.

38. A method for treating a living animal body, including a human, afflicted with an overweight condition characterised by a body mass index greater than 25 and less than 30 caused by treatment for type I or II diabetes comprising the step of administering to the living animal body, including a human, an amount of a compostion of claim 26 which is effective for alleviation of the condition.

39. A method for treating and/or preventing a living animal body, including a human, afflicted with obesity, comprising the step of administering to the living animal body, including a human, an amount of a composition comprising one or more PPAR ligands and an antioxidant agent which is effective for alleviation of obesity.

40. A method for treating and/or preventing a living animal body, including a human, afflicted with obesity caused by a therapeutic treatment, comprising the step of administering to the living animal body, including a human, an amount of a composition comprising one or more PPAR ligands and an antioxidant agent which is effective for alleviation of obesity caused by a therapeutic treatment.

41. A method for treating and/or preventing a living animal body, including a human, afflicted with obesity caused by treatment for type I or II diabetes, comprising the step of administering to the living animal body, including a human, an amount of a composition comprising one or more PPAR ligands and an antioxidant agent which is effective for alleviation of obesity caused by treatment for type I or II diabetes.

42. A method for treating and/or preventing a living animal body, including a human, afflicted with an overweight condition characterised by a body mass index greater than 25 and less than 30, comprising the step of administering to the living animal body, including a human, an amount of a composition comprising one or more PPAR ligands and an antioxidant agent which is effective for alleviation of the condition.

43. A method for treating and/or preventing a living animal body, including a human, afflicted with an overweight condition characterised by a body mass index greater than 25 and less than 30 caused by a therapeutic treatment, comprising the step of administering to the living animal body, including a human, an amount of a composition comprising one or more PPAR ligands and an antioxidant agent which is effective for alleviation of the condition.

44. A method for treating and/or preventing a living animal body, including a human, afflicted with an overweight condition characterised by a body mass index greater than 25 and less than 30 caused by treatment for type I or II diabetes, comprising the step of administering to the living animal body, including a human, an amount of a composition comprising one or more PPAR ligands and an antioxidant agent which is effective for alleviation of the condition.
